(19) United States
(12) Patent Application Publication
(10) Pub. No.: US 2018/0165615 A1
(43) Pub. Date: Jun. 14, 2018

(54) DISPLAY FOR ANALYZING AND OPTIMIZING MEDICAL RESOURCE CONSUMPTION

(71) Applicant: hMetrix LLC, Bala Cynwyd, PA (US)

(72) Inventors: George Chalissery, Bala Cynwyd, PA (US); Verghese Verghese, Cochin (IN); Jineesh Kachira James, Cochin (IN); Sindhumol Sabu, Cochin (IN); Afsal Tharupeedikayil Abdulkhader, Cochin (IN); Udaya Adukkadukkam, Cochin (IN); Sreekumar Mullasseril Sasidharan, Cochin (IN)

(21) Appl. No.: 15/860,844

(22) Filed: Jan. 3, 2018

Related U.S. Application Data
(63) Continuation-in-part of application No. 15/187,419, filed on Jun. 20, 2016, now abandoned.

(51) Int. Cl. G06Q 10/06; G06F 17/30; G16H 70/20; G16H 10/60

(52) U.S. Cl. CPC G06Q 10/0631; G06F 17/30; G16H 40/20; G16H 10/60; G16H 70/20

(57) ABSTRACT

An interactive display may be configured to provide inpatient data for analysis and optimization of medical resource consumption. The inpatient data may be associated with cost information for one or more service matters for one or more discharges.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method of optimization of medical resource consumption by receiving inpatient data directed to cost information for one or more service matters for one or more discharges. For each discharge, the method may classify the received inpatient data into one or more diagnosis related groups (DRG)s. The method may then generate one or more clusters of physicians. The clusters may be generated via a k-means method, where the clusters of physicians is based on a consumption of the one or more service matters for each of the plurality of DRGs. The clusters may be generated iteratively to determine an optimal number of clusters based on an overall r-square value, where one or more responsible physicians (RP)s with identical consumption across the one or more service matters within each of the plurality of DRGs are assigned to the same cluster. If the received inpatient data contains cost information at a service item level, for each consumption of the one or more service matters with one of the plurality of DRGs, the method may determine one or more conflict indexes across the clusters of physicians based on a cost incurred for consumption of the one or more service matters. The method may receive feedback from a remote user computer and generate a visualization based on the feedback. The method may transmit the visualization to interactively display on the remote user computer display, in an interactive dashboard. The interactive dashboard may include the one or more conflict indexes for each of the plurality of DRGs and the one or more service matters having the greatest potential for cost savings, where user interaction with the interactive dashboard displaying the one or more conflict indexes causes display of the consumption across the one or more service matters for each of the discharges for each of the one or more clusters. Other embodiments of this aspect include corresponding computer systems, apparatus, and computer programs recorded on one or more computer storage devices, each configured to perform the actions of the methods.

Implementations may include one or more of the following features. For example, the method may be where the inpatient data is inpatient data for one hospital or inpatient data for multiple hospitals in a particular grouping. The method where, if the received inpatient data includes charge information and does not contain cost information at a service item level, the charge information is adjusted to costs using cost to charge ratios (RCC)s while excluding or correcting outlier RCCs and missing RCCs to form a costed discharge record as the received inpatient data. The method where the RP is assigned to a cluster if the number of cases that the RP takes care for the same DRG is more than a threshold minimum cases number (nc), and if the number of RPs taking care of one DRG is more than the threshold minimum physicians number (np). The method may further include determining a potential cost savings opportunity where the potential cost savings opportunity is a variance between an actual cost and an optimal cost based on resource consumption. The method may further include interactively displaying the potential cost savings opportunities on the remote user computer display. The method may further include classifying the plurality of DRGs with an all patient refined diagnosis related group (APR DRG). The method where if the APR DRG is surgical, an RP is a first entry in another physician location of a uniform bill, and if the other physician location is empty, an attending physician is used as the RP. If the attending physician is empty, no RP is assigned. If the APR DRG is not surgical, the RP is an attending physician, and if the attending physician is empty, no RP is assigned. Implementations of the described techniques may include hardware, a method or process, or computer software on a computer-accessible medium.

One general aspect includes a system for optimization of medical resource consumption. The system may include a computer that includes a computer processor configured to receive inpatient data from a remote computer. The inpatient data may be directed to cost information for one or more service matters for one or more discharges. The computer processor may be further configured to classify, for each of the discharges, the received inpatient data into a plurality of DRGs. The computer processor may also be configured to generate one or more clusters of physicians via a k-means method, where the clusters of physicians is based on a consumption of the one or more service matters for each of the plurality of DRGs. The computer processor may also be configured to generate the plurality of clusters iteratively to determine an optimal number of clusters based on an overall r-square value, where a plurality of RPs with identical consumption across the one or more service matters within each of the plurality of DRGs are assigned to the same cluster and for each consumption of the one or more service matters with one of the plurality of DRGs. If the received inpatient data contains cost information at a service matter level, the computer processor may determine a plurality of conflict indexes across the clusters based on a cost incurred for consumption of the one or more service matters. The computer processor may be configured to receive feedback from a remote user computer. The computer processor may be configured to generate a visualization based on the feedback. The computer processor may be configured to transmit the visualization to interactively display on the remote user computer display, in an interactive dashboard. The interactive dashboard may include the conflict indexes for each of the DRGs and the one or more service matters having the greatest potential for cost savings. User interaction with the interactive dashboard displaying the conflict indexes may cause the display of the consumption across the one or more service matters for each of the discharges for each of the one or more clusters. Other embodiments of this aspect include corresponding computer systems, apparatus, and computer programs recorded on one or more computer storage devices, each configured to perform the actions of the methods.

Implementations may include one or more of the following features. For example, the computer processor may use inpatient data that is inpatient data for one hospital or inpatient data for multiple hospitals in a particular grouping. If the received inpatient data includes charge information and does not contain cost information at a service item level, the charge information may be adjusted to costs using RCCs while excluding or correcting outlier RCCs and missing RCCs to form a costed discharge record as the received inpatient data. The computer processor may be configured to assign the RP to a cluster if the number of cases that the RP takes care for the same DRG is more than a threshold minimum cases number (nc), and if the number of RPs taking care of one DRG is more than the threshold minimum physicians number (np). The computer processor may be further configured to determine a potential cost savings opportunity where the potential cost savings opportunity is a variance between an actual cost and an optimal cost based on resource consumption. The computer processor may be further configured to interactively display the potential cost savings opportunities on the remote user computer display. The computer processor may be further configured to classify the plurality of DRGs with an APR DRG. If the APR DRG is surgical, an RP may be a first entry in another physician location of a uniform bill. If the other physician location is empty, an attending physician is used as the RP, and if the attending physician is empty, no RP is assigned. If the APR DRG is not surgical, the RP is an attending physician, and if the attending physician is empty, no RP is assigned. Implementations of the described techniques may include hardware, a method or process, or computer software on a computer-accessible medium.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a flow diagram of a method of evaluating data of medical resource consumption by physicians and interactively displaying potential cost savings based on the evaluated data.

FIG. 2 is a flow diagram of a method for assigning Diagnosis Related Group (DRG) to discharges and data preparation of the assigned grouping.

FIG. 3 is a flow diagram of a method of clustering physicians.

FIG. 4 is a flow diagram of a method for calculating the conflict index for each consumption dimension within one DRG.

FIGS. 5A and 5B are a flow diagram of a method for calculating the potential saving opportunity.

FIG. 6 depicts a system diagram outlining the physical systems relationships between a medical facility data system, a data center and an end user.

FIGS. 7A and 7B are an example of an interactive user dashboard displaying potential cost savings based on the evaluated data.

FIGS. 8A through 8D are examples of different reports displaying savings opportunities by the top APR DRGs and by the top physicians based on the evaluated data.

### DETAILED DESCRIPTION

Reference will now be made in greater detail to a preferred embodiment of the invention, an example of which is illustrated in the accompanying drawings. Wherever possible, the same reference numerals will be used throughout the drawings and the description to refer to the same or like parts.

As used herein, the terminology “computer” or “computing device” includes any unit, or combination of units, capable of performing any method, or any portion or portions thereof, disclosed herein.

As used herein, the terminology “processor” indicates one or more processors, such as one or more special purpose processors, one or more digital signal processors, one or more microprocessors, one or more controllers, one or more microcontrollers, one or more application processors, one or more central processing units (CPU)s, one or more graphics processing units (GPU)s, one or more digital signal processors (DSP)s, one or more application specific integrated circuits (ASIC)s, one or more application specific standard products, one or more field programmable gate arrays, any other type or combination of integrated circuits, one or more state machines, or any combination thereof.

As used herein, the terminology “memory” indicates any computer-usable or computer-readable medium or device that can tangibly contain, store, communicate, or transport any signal or information that may be used by or in connection with any processor. For example, a memory may be one or more read only memories (ROM), one or more random access memories (RAM), one or more registers, low power double data rate (LPDDR) memories, one or more cache memories, one or more semiconductor memory devices, one or more magnetic media, one or more optical media, one or more magneto-optical media, or any combination thereof.

As used herein, the terminology “instructions” may include directions or expressions for performing any method, or any portion or portions thereof, disclosed herein, and may be realized in hardware, software, or any combination thereof. For example, instructions may be implemented as information, such as a computer program, stored in memory that may be executed by a processor to perform any of the respective methods, algorithms, aspects, or combinations thereof, as described herein. Instructions, or a portion thereof, may be implemented as a special purpose processor, or circuitry, that may include specialized hardware for carrying out any of the methods, algorithms, aspects, or combinations thereof, as described herein. In some implementations, portions of the instructions may be distributed across multiple processors on a single device, on multiple devices, which may communicate directly or across a network such as a local area network, a wide area network, the Internet, or a combination thereof.

As used herein, the terminology “determine” and “identify,” or any variations thereof, includes selecting, ascertaining, computing, looking up, receiving, determining, establishing, obtaining, or otherwise identifying or determining in any manner whatsoever using one or more of the devices and methods shown and described herein.

As used herein, the terminology “example,” “embodiment,” “implementation,” “aspect,” “feature,” or “element” indicates serving as an example, instance, or illustration. Unless expressly indicated, any example, embodiment, implementation, aspect, feature, or element is independent of each other example, embodiment, implementation, aspect, feature, or element and may be used in combination with any other example, embodiment, implementation, aspect, feature, or element.

As used herein, the terminology “or” is intended to mean an inclusive “or” rather than an exclusive “or.” That is, unless specified otherwise, or clear from context, “X includes A or B” is intended to indicate any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from the context to be directed to a singular form.

As used herein, the terminology “consumption” includes utilization, operation, use, application, exploitation and any of the natural inclusive permutations.

Further, for simplicity of explanation, although the figures and descriptions herein may include sequences or series of steps or stages, elements of the methods disclosed herein may occur in various orders or concurrently. Additionally, elements of the methods disclosed herein may occur with other elements not explicitly presented and described herein. Furthermore, not all elements of the methods described herein may be required to implement a method in accordance with this disclosure. Although aspects, features, and elements are described herein in particular combinations, each aspect, feature, or element may be used independently or in various combinations with or without other aspects, features, and elements.

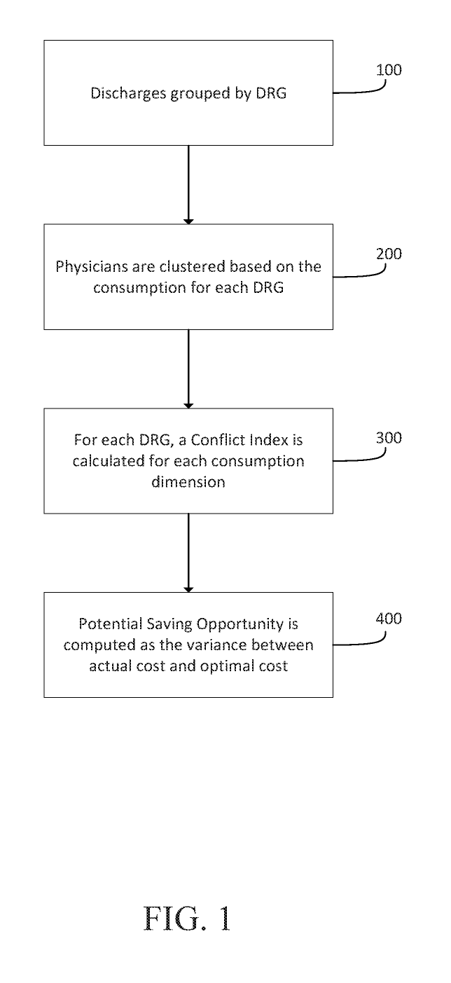

FIG. 1 is a flow diagram a method of evaluating data of medical resource consumption by physicians and interactively displaying potential cost savings based on the evaluated data. In block 100, discharge data is processed and grouped by Diagnosis Related Group (DRG). Physicians are then clustered in block 200 based on the consumption for each DRG. In block 300, for each DRG, a conflict index is calculated for each consumption dimension. In block 400, a Potential Saving Opportunity is computed as the variance between an actual cost and an optimal cost.

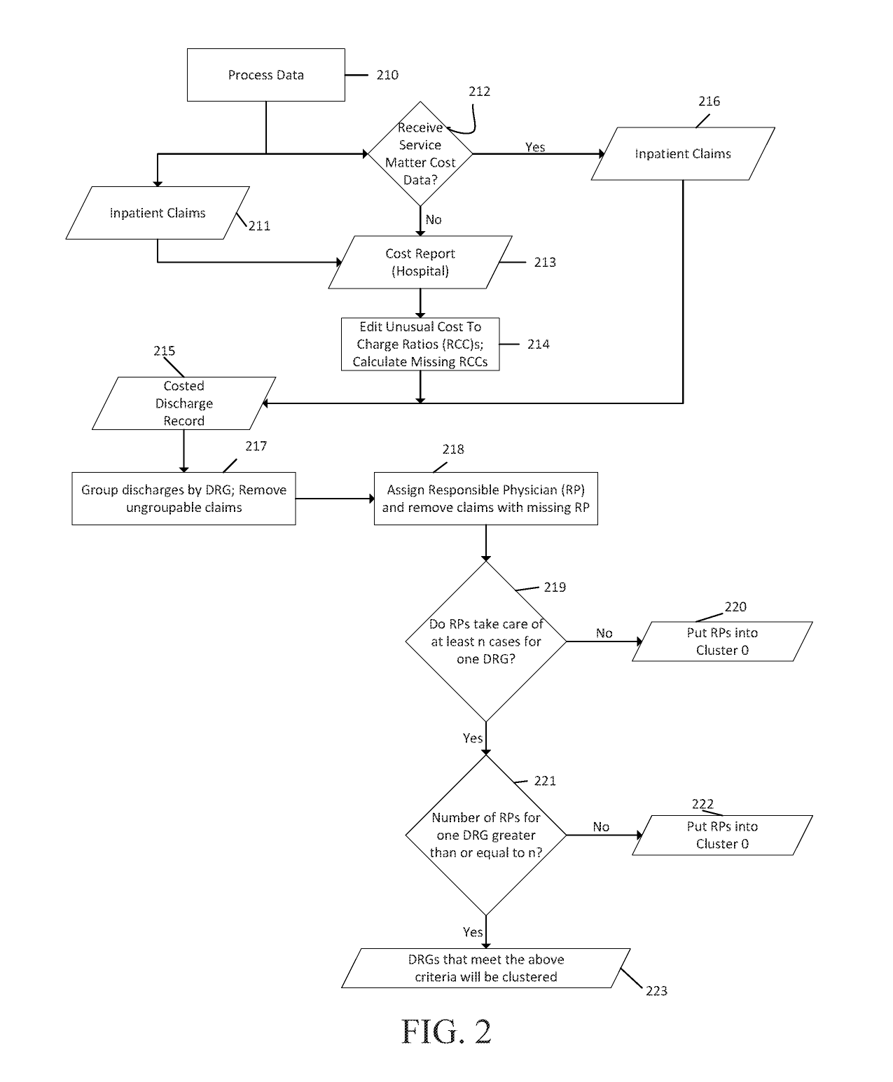

FIG. 2 is a flow diagram of an implementation of block 100 including a method for assigning DRGs to discharges and steps of data preparation. In block 210, medical data is obtained. The medical data may be obtained from a remote computer, for example a medical facility data system. The medical data can be all inpatient data for one hospital or a plurality of hospitals in a particular grouping. For example, the inpatient data grouping can relate to all inpatient data of all hospitals in one healthcare system. Alternatively, the inpatient data grouping can relate to inpatient data of hospitals in a portion of a state, such as hospitals in a particular county or a selected group of participating hospitals.

In block 211, inpatient claim data is determined from inpatient claim information of the obtained medical data which is generated during inpatient stays at hospitals or the like. The inpatient claim information includes all claims associated with the patient's stay in the hospital, such as for example room and board, prescription drug claims, medical tests and the like. Inpatient claim information can be derived from claim information entered on conventional forms such as the uniform/universal billing Form 8371 or Form CMS-1450, also known as UB-04, which is the official HCFA/CMS form used by hospitals and health care centers when submitting bills to Medicare and 3^{rd}-party payors for reimbursement for health services provided to patients covered or any other similar methods of collecting claim information that are conventionally used by hospitals.

In block 212 it is determined if the inpatient claim data contains cost information at a service matter level from a hospital. A service matter may also be referred to as a service item. A service matter can be a matter used as part of the care delivered for each discharge at the hospital. The service matter can include one or more matters used in different aspects or revenue centers of the hospital. Examples of service matters include individual medical supply items, surgical supply items, drugs, laboratory, radiology, operating room and room & board items. If the inpatient claim data contains cost information at a service matter level from a hospital, then block 216 is implemented. If the inpatient claim data does not contain cost information at a service matter level from a hospital, then block 213 is implemented. In block 213, an inpatient cost to charge ratio data is determined from cost reports, such as the Medicare hospital cost reports. In block 214, the cost report data is edited to exclude or correct outlier cost to charge ratios (RCC) and a missing RCC ratio is calculated using a statistical method. In one embodiment, a regression model is run using the statewide data to come up with the RCC ratio for the cost centers without RCC ratio. In block 215, the costs incurred per inpatient claim are determined from the discharge claim information of block 216 and the cost report data to form a costed discharge record. For example, the costs can be determined by industry standard cost accounting techniques, such as hospital-specific, cost-center-specific and ratio of costs to charges.

In block 217, the costed discharge record along with the inpatient services provided for them are classified using groupings of Diagnosis Related Group (DRG)s. The classification of the diagnosis related groups can be adjusted for severity of illness. In the adjustment for severity of illness, the DRGs can be further defined by describing each diagnosis in terms of four levels of medical severity referred to as refinement classes. For example, refinement classes can include whether the DRG is a grouping of medical or surgical diagnoses, the patient's sex, the patient's age, whether the patient died within two days of admission, and whether the patient was discharged against medical advice. For example, for a hip replacement surgery, an obese 70-year-old patient with diabetes and a liver transplant is likely to place a greater drain on resources versus a fit 70 year old patient looking for a hip replacement. Even though these discharges fall into the same DRG, the cost attributed to the treatment of each can be more accurately analyzed due to the refining of the DRG. In this manner, refined DRGs group discharges according to resource intensity, and thus allow more accurate comparisons. For example, block 217 can be implemented for classifying Medicare fee-for-service inpatient stays by determining ALL PATIENT REFINED DIAGNOSIS RELATED GROUPS (APR DRGs) using Averill, R. F. et al. Definition Manual, 3M Health Information System, Wallingford, Conn., 1988, hereby incorporated by reference into this application and as described in U.S. Pat. No. 5,652,842 hereby incorporated in its entirety by reference into this application, can be used to determine classified diagnosis related groups. It will be appreciated that in the present disclosure, classified DRGs are referred to as APR DRG and that APR DRGs can refer to classified DRGs which can be determined by other discharge classification methods. Claim records with Ungroupable APR DRGs are removed from further analysis.

In block 218, the classified services provided to a discharge are assigned to a responsible physician (RP). An RP is defined as the physician most responsible for resource consumption while the patient is hospitalized. In the APR DRG grouping, all inpatient facility claims are classified as either medical or surgical. The following two physician fields on the conventional uniform/universal billing Form 8371 or Form CMS-1450, also known as UB-04, can be used in the RP determination process: Attending Physician referenced by Form Locator 76 and other physician referenced by Form Locator 77. For example, the operating physician can be the surgeon.

An example method for the determination of the RP is as follows:

1) If the APR DRG is surgical, the RP is the first entry in the other physician location. If the other physician location is empty, the attending physician is used;

2) If the APR DRG is not surgical, the RP is the attending physician;

3) If the attending physician is empty, then no RP is assigned. These claims records are removed from further analysis.

In block 219, it is determined if the RP takes care of a threshold number of cases for the same DRG. Accordingly, if the number of cases that the RP takes care for the same DRG is less than the threshold minimum cases number (nc), the RP is assigned to Cluster 0 in block 220.

In block 221, it is determined if a minimum number of RP take care of one DRG. Accordingly, if the number of RPs taking care of one DRG is less than the threshold minimum physicians number (np), the DRG is assigned to Cluster 0 in block 222. In block 223, the dataset of determined DRGs meeting the criteria is created.

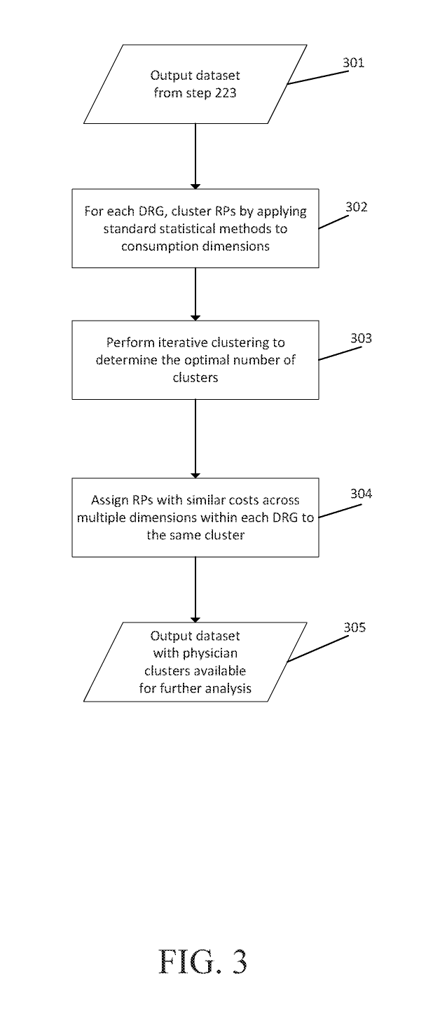

Referring to FIG. 1, in block 200 physicians are clustered using the dataset of determined DRGs meeting the criteria. FIG. 3 is a flow diagram of an implementation of block 200 for a method of clustering physicians.

In block 301, the dataset of determined DRGs meeting the criteria from block 223 is input in block 302, for each DRG, RPs are clustered by applying conventional statistical methods, such as for example a k-means method, to consumption dimensions of the DRG. Consumption dimensions refer to the broad categorization of the items utilized by physicians to treat patients. For example, medical/surgical supplies, drugs, labs and radiology, etc. All resources of the DRG are considered when performing the clustering. For example, physicians with discharges in the same DRG who are identical in room and board consumption, but different in the use of drugs will be placed in different clusters. Physicians within a determined cluster are similar to each other, while physicians in different clusters are different from each other.

In block 303, for each DRG, clustering is done iteratively to decide the optimal number of clusters based on the overall R-Square value. The number of clusters is typically limited to 2-5. For different APR DRGs, the number of clusters can be different. For example, for an APR DRG for Extreme Septicemia and Disseminated Infections, the optimal number of clusters may be determined to be 3 clusters while for an APR DRG for Major Chronic Obstructive Pulmonary Disease, the optimal number of clusters may be determined to be 4 clusters. A physician can only belong to one cluster. As a result, RPs with similar costs across multiple dimensions within each DRG are assigned to the same cluster in block 304. In block 305, the data set of RP clusters is created.

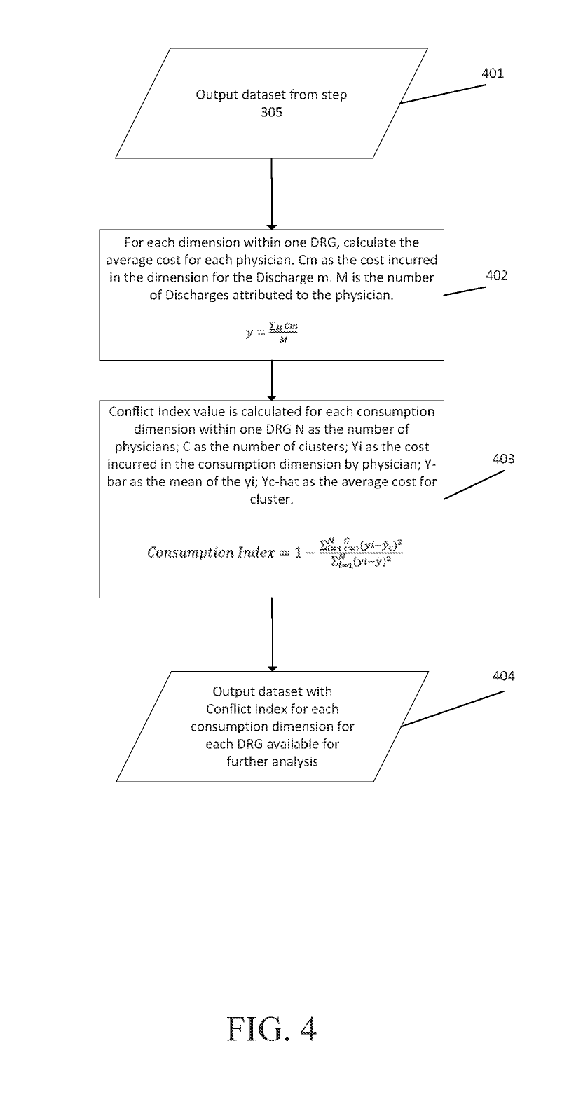

Referring to FIG. 1, in block 300 the dataset of RP clusters is used to calculate a conflict index for each consumption dimension for each DRG. A conflict index may also be referred to as a difference index. FIG. 4 is a flow diagram of an implementation of block 300 for calculating the conflict index for each consumption dimension within one DRG.

In block 401, the dataset of RP clusters determined from block 305 is input.

In block 402, for each dimension within one DRG, the average cost for each physician is calculated. It is calculated as the average cost per discharge across all discharges within each APR DRG that is attributed to the RP. Cm is the cost incurred in the dimension for the discharge m. M is the number of discharges attributed to the physician. At step 402, for each dimension within one DRG, for each of the RPs, average cost (y) is calculated where

$$y = \frac{{\sum }_{M} Cm}{M}$$

In block 403, a Conflict Index value is calculated for each consumption dimension within one DRG. N is the number of RPs for that DRG. C is the number of clusters. Within one DRG, for one consumption dimension, Yi is the average cost for an RP i calculated in step 402; Y-bar is the mean of the average cost among all the physicians for the DRG; Yc-hat is the average physician cost in cluster c. At this step, the Conflict Index is calculated where

$$Conflict Index = 1 - \frac{{\sum }_{{i = 1}_{c = 1}}^{{N}^{C}} {( yi - {\tilde{y}}_{c} )}^{2}}{{\sum }_{i = 1}^{N} {( yi - \overline{y} )}^{2}}$$

In block 404, the data set of a Conflict Index for each consumption dimension for each DRG is created.

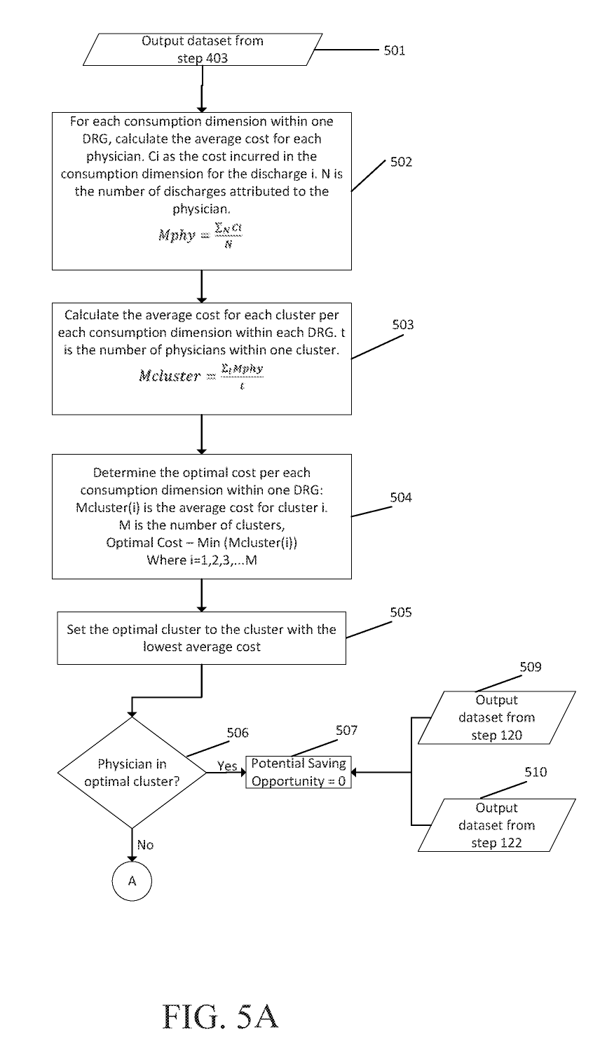

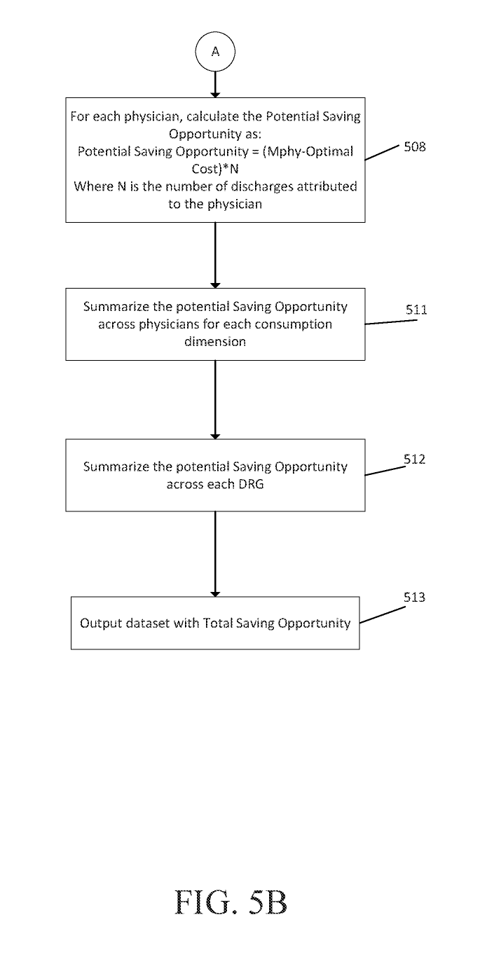

Referring to FIG. 1, in block 400 the data sets of average actual discharge cost by a physician and the physician clustering result are used to determine a potential cost savings opportunity. A potential cost savings opportunity is the variance between the actual discharge cost and the optimal discharge cost. FIGS. 5A and 5B are a flow diagram of an implementation of block 400 for calculating the potential cost saving opportunity.

In block 501, the data set of a Conflict Index for each consumption dimension for each DRG is input.

In block 502, for each dimension within one DRG, the average cost for each physician is calculated. It is calculated as the average cost per discharge across all discharges within each APR DRG that is attributed to the RP. Ci is the cost incurred in the dimension for the Discharge i. N is the number of discharges attributed to the physician. At this step, for each dimension within one DRG, for each RP, average cost (Mphy) is calculated where

$$Mphy = \frac{{\sum }_{N} Ci}{N}$$

In block 503, the average cost for each cluster per each consumption dimension within each DRG is calculated. It is calculated as the mean of the average cost among all the physicians in the cluster. t is set as the number of physicians within one cluster. At this step, average cost for each cluster is calculated where

$$Mcluster = \frac{{\sum }_{t} Mphy}{t}$$

In block 504, the optimal cost per each consumption dimension within one DRG is determined. The optimal cost is the minimum average cost among all the clusters. Mcluster(i) is set as the average cost for cluster i calculated in step 503. M is the number of clusters. At this step, the optimal cost is calculated where

Optimal Cost=Min(Mcluster(i))i=1,2,3 . . . M

In block 505, within one DRG, the optimal cluster is set to the cluster with the minimum average cost among clusters which is determined in step 504.

In block 506, within one DRG, in order to calculate the potential saving opportunity for each of the RPs, first determine if the RP is in the optimal cluster which was set in step 505. Accordingly, if the RP is determined to be included in the optimal cluster, then a potential saving opportunity for this RP is set as 0 in block 507. Otherwise, in block 508, the potential saving opportunity for this RP is set as the variance between the Actual Cost and the Optimal Cost calculated in block 504. The Potential Saving Opportunity is calculated in step 508 as

Potential Saving Opportunity=(Mphy−Optimal Cost)*N

where N is the number of discharges attributed to the physician and Mphy was calculated in step 502.

In block 509, the output dataset from step 220 is input and the potential saving opportunity for the physicians included is set as 0 in block 507.

In block 510, the output dataset from step 222 is input and for the included DRGs, the potential saving opportunity for the physicians included is set as 0 in block 507.

In block 511, within one DRG, the Potential Saving Opportunity is summarized across physicians for each consumption dimension. In block 512, summarize the Potential Saving Opportunity in each DRG.

In block 513, final output dataset with Total Saving Opportunity is created.

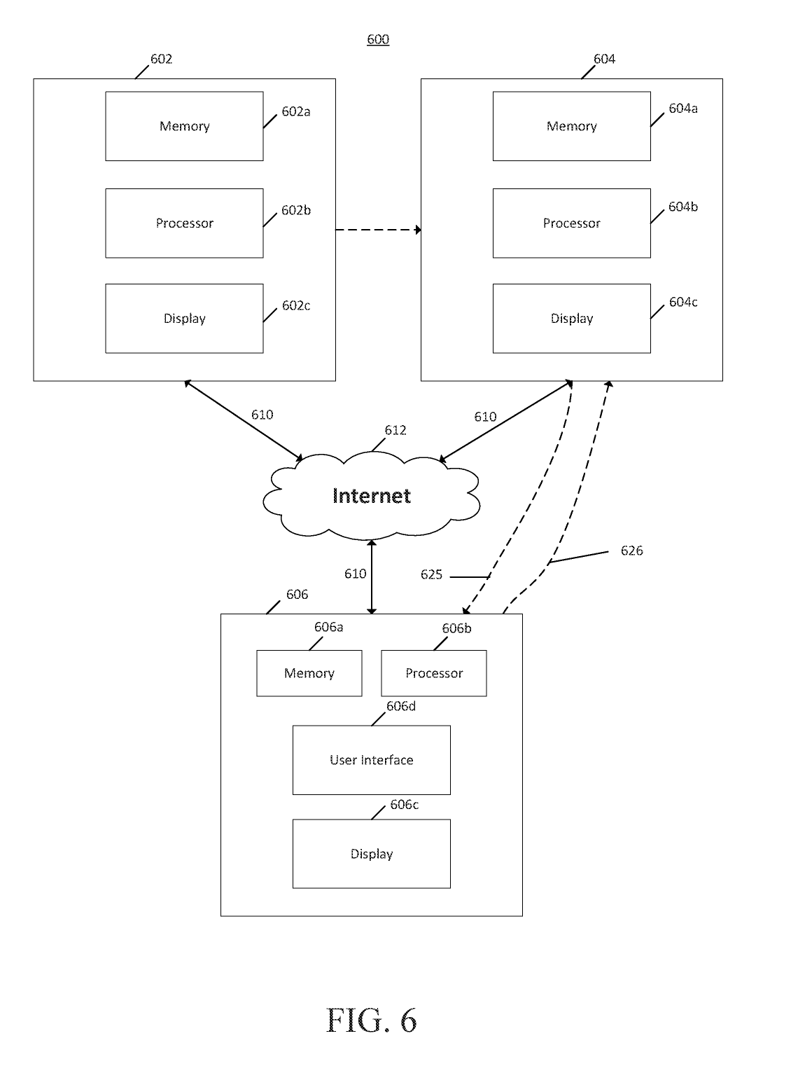

FIG. 6 depicts a schematic diagram of system 600 including a remote computer 602, data center computer 604 and an end user computer 606 and the flow of information throughout the physical system. The remote computer 602 may be a medical facility data system. The end user computer 606 may also be referred to as a remote user computer. For simplicity, one end user computer 606 is shown. It is understood that multiple end user computers and multiple locations may be implemented.

The remote computer 602 includes a memory 602a, a processor 602b, and a display 602c. Some implementations of the remote computer 602 may not include the display 602c. The memory 602a may include a system memory module that is configured to store executable computer instructions that, when executed by processor 602b, control various functions of the remote computer 602. The memory 602a may include non-transitory memory configured to store clinical discharge data and financial data. The processor 602b may include a system on a chip (SOC) microcontroller, microprocessor, CPU, DSP, ASIC, GPU, or other processors that control the operation and functionality of the remote computer 602. The processor 602b may interface with mechanical, electrical, sensory, and power modules via driver interfaces and software abstraction layers. Additional processing and memory capacity may be used to support these processes. These components may be fully controlled by the processor 602b. In some implementations, one or more components may be operable by one or more other control processes in accordance with a given schedule.

The data center computer 604 includes a memory 604a, a processor 604b, and a display 604c. Some implementations of the data center computer 604 may not include the display 604c. The memory 604a may include a system memory module that is configured to store executable computer instructions that, when executed by processor 604b, control various functions of the remote computer 604. The memory 604a may include non-transitory memory configured to store clinical discharge data and financial data received from the remote computer 602. The processor 604b may include a system on a chip (SOC) microcontroller, microprocessor, CPU, DSP, ASIC, GPU, or other processors that control the operation and functionality of the data center computer 604. The processor 604b may interface with mechanical, electrical, sensory, and power modules via driver interfaces and software abstraction layers. Additional processing and memory capacity may be used to support these processes. These components may be fully controlled by the processor 604b. In some implementations, one or more components may be operable by one or more other control processes in accordance with a given schedule.

The end user computer 606 includes a memory 606a, a processor 606b, a display 606c, and a user interface 606d. In some embodiments, the display 606c and user interface 606d may be combined into a single element, for example, a touchscreen display. The memory 606a may include a system memory module that is configured to store executable computer instructions that, when executed by processor 606b, control various functions of the end user computer 606. The memory 606a may include non-transitory memory configured to store visualizations received from the data center computer 604. The processor 606b may include a system on a chip (SOC) microcontroller, microprocessor, CPU, DSP, ASIC, GPU, or other processors that control the operation and functionality of the end user computer 606. The processor 606b may interface with mechanical, electrical, sensory, and power modules via driver interfaces and software abstraction layers. Additional processing and memory capacity may be used to support these processes. These components may be fully controlled by the processor 606b. In some implementations, one or more components may be operable by one or more other control processes in accordance with a given schedule. The display 606c may be configured to provide information related to clinical data and financial data from a medical facility data system. In some implementations, the user interface 606d may include virtually any device capable of registering inputs from and communicating outputs to a user. These may include, without limitation, display, touch, gesture, proximity, light, sound receiving/emitting, wired/wireless, and/or other input/output devices. The user interface 606d may include the display 606c, and one or more tactile elements (e.g., keyboard, joystick, mouse, switch, button, and/or virtual touch screen buttons). The user interface 606d may be configured to enable the user to provide feedback to the data center computer 604 to receive a customizable, dynamic, and interactive visualization to be displayed on display 606c.

Clinical discharge data and financial data 610 flow from the remote computer 602 to the data center computer 604 via Internet 612. The data center computer 604 is configured to receive transferred clinical discharge data and financial data 610 at processor 604b. In some embodiments, the clinical discharge data and financial data 610 may be directly transmitted from the remote computer 602 to the data center computer 604. For example, clinical discharge data and financial data 610 can be transferred by processor 602b using FTP data transfer to processor 604b. The data center computer 604, in conjunction with processor 604b, is configured to processes the transferred clinical discharge data and financial data 610. For example, processor 604b may be configured to generate visualized analysis reports 625. The visualized analysis reports 625 are user-configurable and interactive, and may also be referred to as visualizations. The visualizations may be accessible to multiple users without having to re-process the data. This system also allows for customizable views and real-time updates. The visualizations allow different viewers to view the data in different ways.

Memory 604a may be a database that is configured to store information from processor 604b including the visualized analysis reports 625. Processor 604b can send data such as the visualized analysis reports 625 to the end user computer 606 over internet 612. The end user computer 606 can access visualized analysis reports 625. The end user computer 606, via processor 606b, may transmit user feedback 626 to the data center computer 604 to receive a real-time updated visualization. The user feedback 626 may be in the form of data or commands to cause real-time results or updated information based on the user feedback 626 to be transmitted to the end user computer 606. The end user computer 606 is configured to interactively display the visualized analysis reports 625 such that the user may interact with the received visualization to provide feedback and receive updated results in real-time based on the provided feedback. Interactively displaying may include, filtering, focusing, changing a view, viewing an alternate dataset, changing a visualization type, etc. For example, a user may interact with the visualization by clicking on a portion of the visualization to drill down and receive additional information, for example a focused subset of the visualized analysis report. The user may click on and highlight visualization elements thereby filtering other elements. For example, the user may highlight a specific APR DRG to filter the visualization to only include information about the selected APR DRG. Hovering over a visualization element presents the user with context information. The end user computer 606 may be a desktop computer, a laptop computer, a tablet, or any like device. The display 606c can display the visualized analysis reports 625, for example, as a webpage.

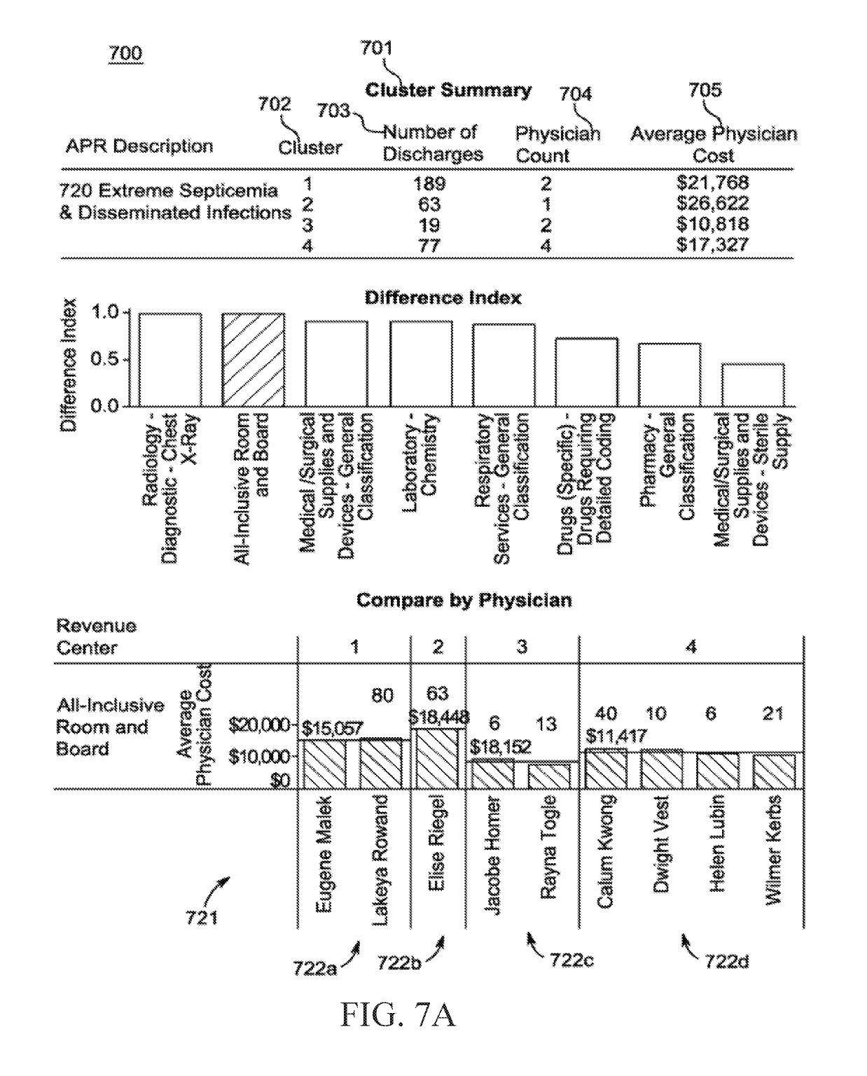

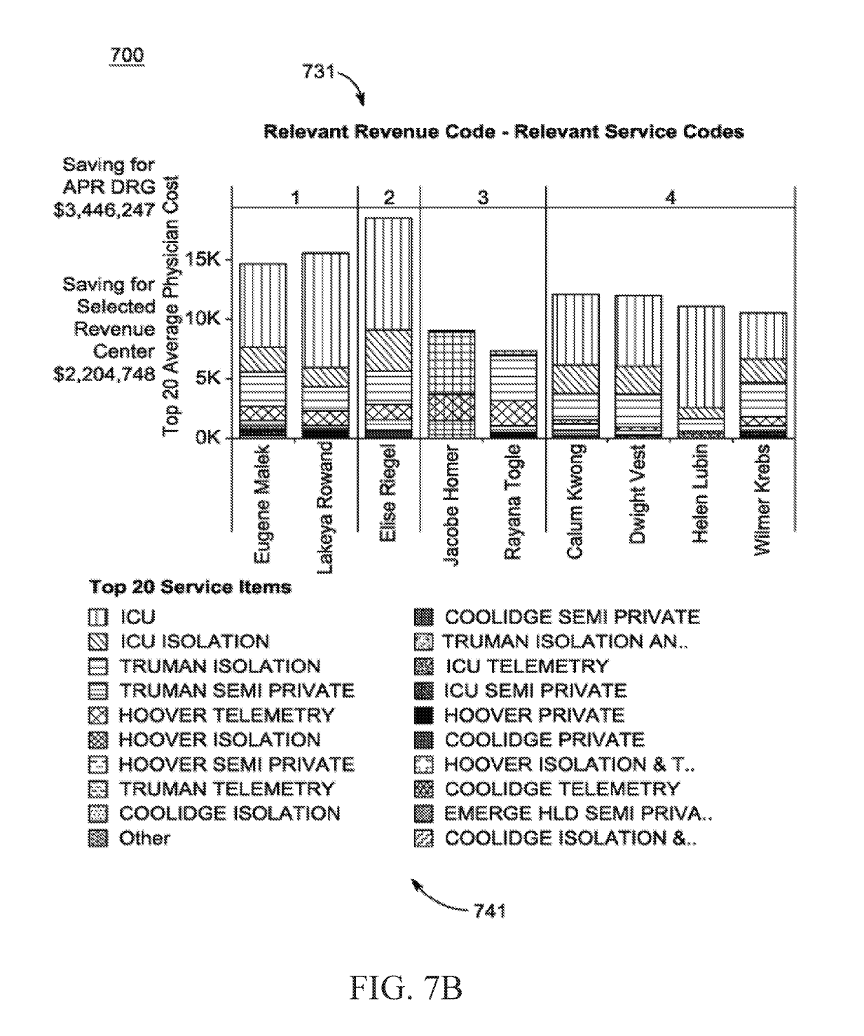

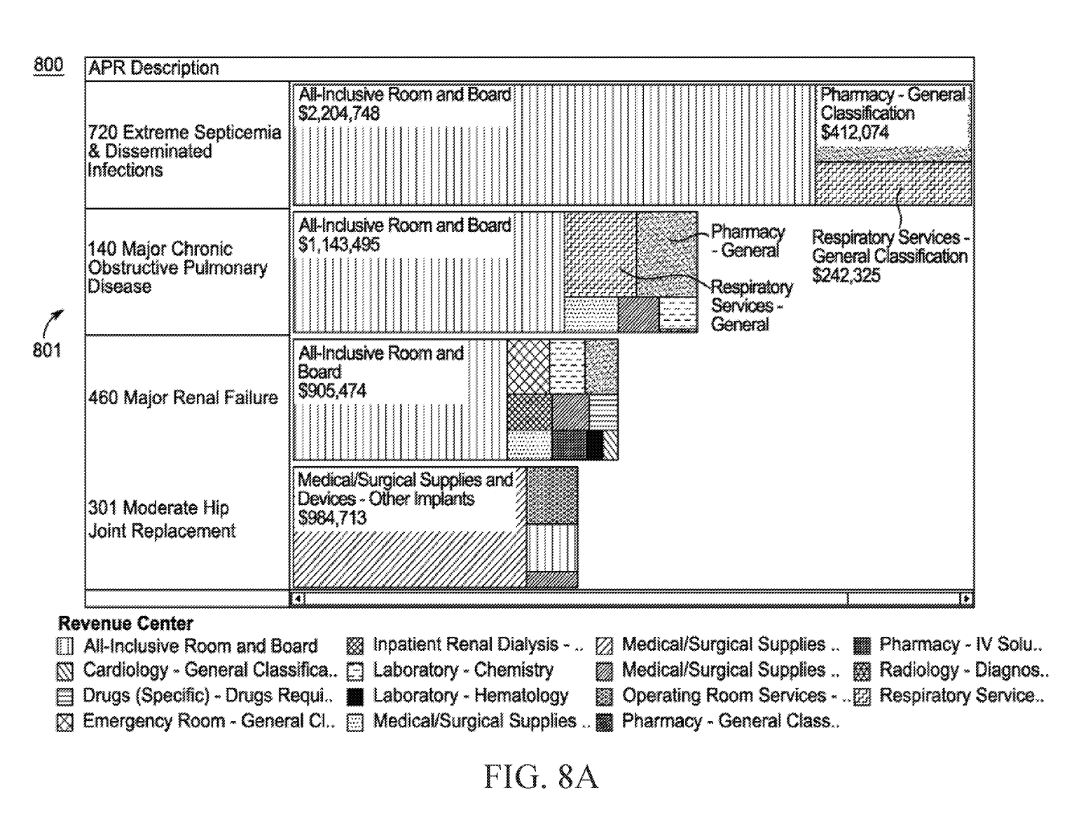

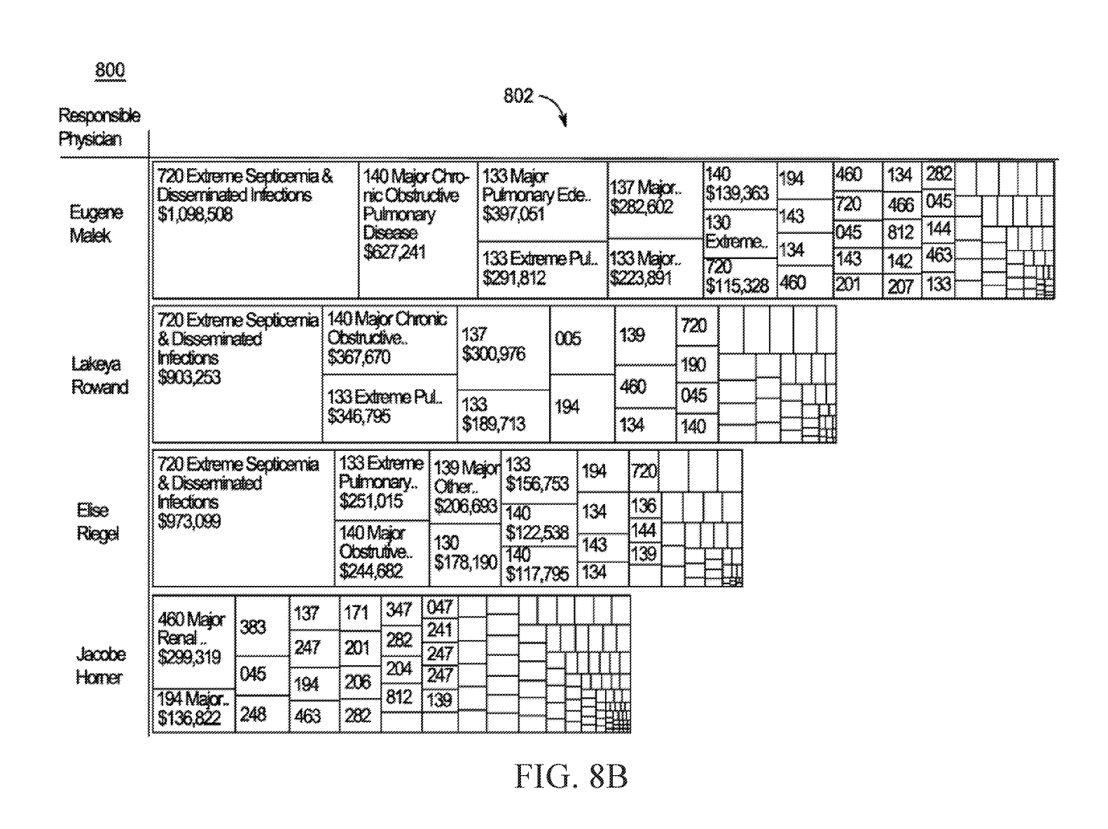

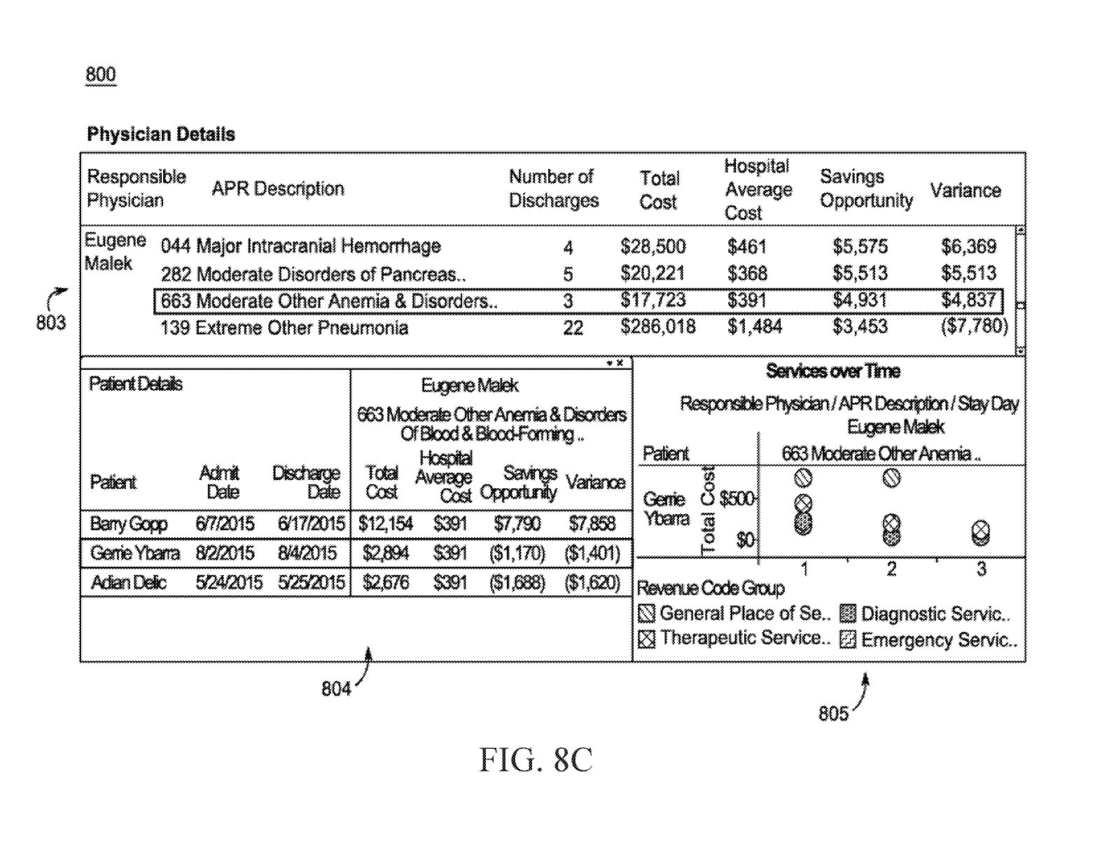

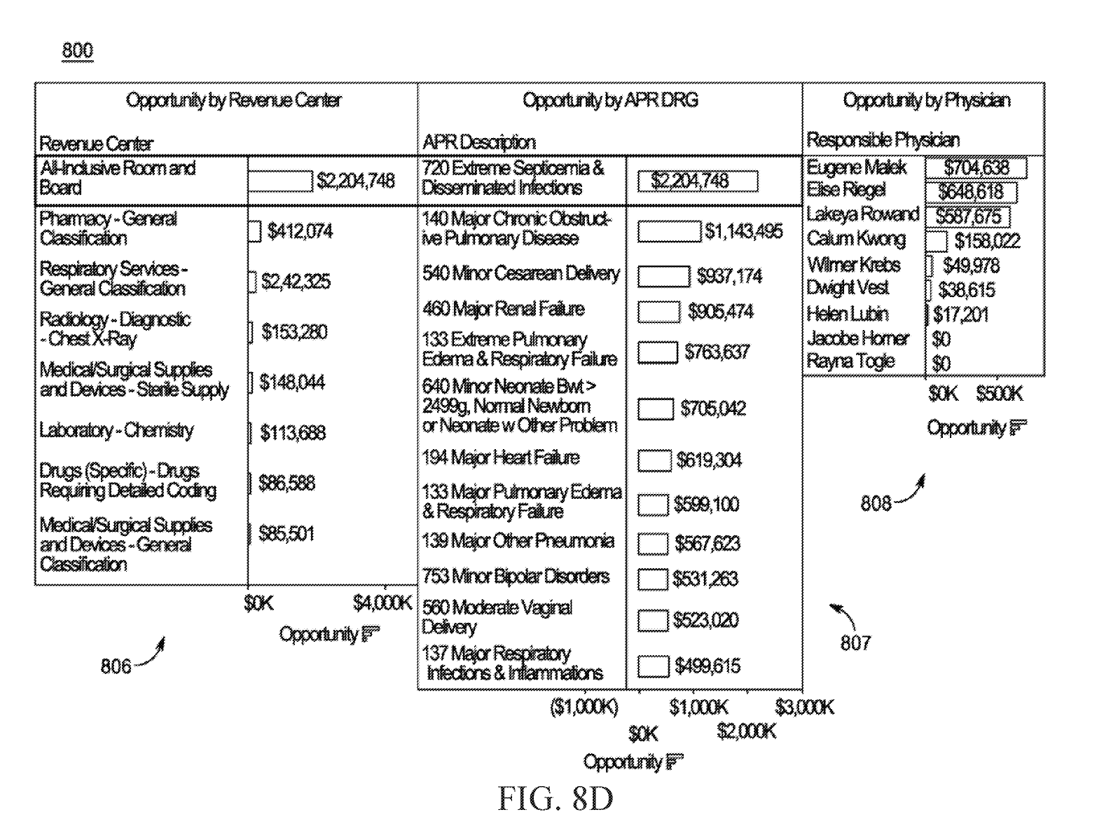

FIGS. 7A and 7B is an example of interactive user dashboard for displaying potential cost savings based on the evaluated data which can be displayed as webpage on display 606c shown in FIG. 6. The interactive user dashboard can be a webpage shown as “DRG dashboard” 700 as shown in FIGS. 7A and 7B. Dashboard 700 displays a cluster summary in portion 701. The cluster summary can include metrics for an APR DRG of a cluster number 702, number of discharges 703, number of RPs 704 and average cost for the APR DRG for each cluster 705. Hovering the mouse cursor over any of these elements presents the user with a pop-out window detailing the data of the cluster such as number of discharges and average cost. Dashboard 700 displays a conflict index summary in portion 711. The conflict index summary can include metrics for a conflict index of a consumption dimension directed to service items 712. Clicking on and selecting any of the revenue centers in the conflict index summary filters the interactive user dashboard to present metrics that only relate to the selected revenue center. Hovering the mouse cursor over any of the conflict indices presents the user with a pop-out window which displays the APR DRG, revenue center, numeric value of the conflict index, and the total cost savings opportunity for the hovered over revenue center. Dashboard 700 can include a comparison of average costs for physician in portion 721. The comparison of average costs for physicians can be shown in respective portions 722a-722d based on assigned clusters. Clicking on any physician in portion 721 presents the user a dashboard that shows detailed information about the selected physician (FIG. 8C). Hovering over any physician in portions 722a-722d displays metrics about the physician such as assigned cluster and average physician cost. Dashboard 700 can display potential cost savings opportunities in portion 731 for each of the responsible physicians (RPs). Hovering over any service item in portion 731 displays the average cost for the specific physician and service item. Dashboard 700 can display the service items having the greatest potential for cost savings in portion 741. Clicking on any of the service items highlights the selected service item in portion 731.

FIGS. 8A through 8D are an example of an interactive user dashboard 800 displaying potential cost savings based on the evaluated data. Interactive user dashboard 800 displays potential saving opportunity for the top APR DRGs by Revenue Centers in portion 801 and top Physicians by APR DRGs in portion 802. Hovering over any element in portions 801 or 802 displays either the revenue center for portion 801 or the APR DRG for portion 802 and the corresponding cost savings. An example of an interactive user dashboard 800 displays cost and potential saving opportunity for physicians by APR DRGs in portion 803, discharge details in portion 804 and services consumed for treating the patients during length of stay in portion 805. Clicking on an APR DRG in portion 803 filters portion 804 to only show those patients who had discharge for the selected APR DRG. Clicking on a patient in portion 804 filters portion 805 with details about the selected patient. Interactive user dashboard 800 can display a total saving opportunity by Revenue Center in portion 806 or by APR DRG in portion 807 or by physicians in portion 808 in an ad-hoc manner. Clicking on a revenue center in portion 806 filters portions 807 and 808 to include cost savings based only on the selected revenue center. Multiple revenue centers can be selected in this way which allows for customized filtering of data for portions 807 and 808. Similarly, clicking on an APR DRG in portion 807 or a physician in portion 808 will filter the other two portions (portions 806 and 808 for clicking on an APR DRG or portions 806 and 807 for clicking on a physician). If a filter is already applied by clicking on an element in portions 806 to 808, selecting an element in an additional portion will further filter the cost savings data based on that selection. For example, selecting a revenue center from portion 806 and an APR DRG from portion 807 will filter based on the selected revenue center and selected APR DRG.

Although some embodiments herein refer to methods, it will be appreciated by one skilled in the art that they may also be embodied as a system or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “processor,” “device,” or “system.” Furthermore, aspects of the present invention may take the form of a computer program product embodied in one or more computer readable mediums having computer readable program code embodied thereon. Any combination of one or more computer readable mediums may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to CDs, DVDs, wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks. The flowcharts and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

A computer implemented method and system for optimization of medical resource utilization within a set of physicians in order to calculate a potential cost savings opportunity is described. Input classified discharge data directed to cost information for service items grouped by a diagnosis related group is assigned to a physician which was most responsible for the resource utilization in treating the patient while the patient was hospitalized. For each diagnosis related group in the classified data, the responsible physicians are dynamically clustered based on resource utilization to identify the factors that are consistently different across the clustered physicians as a difference index value. The difference index value can be analyzed for determining potential cost savings opportunities. An interactive user interface can be used for entering discharge data, dynamically displaying resource utilization by the difference index value and potential cost savings opportunities. The contents of U.S. application Ser. No. 15/187,419 filed on Jun. 20, 2016 are hereby incorporated by reference as if fully set forth.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method of optimization of medical resource consumption, the method comprising:
receiving, by a computer processor, from a remote computer, inpatient data directed to cost information for one or more service matters for a plurality of discharges;
for each of the plurality of discharges, classifying, by the computer processor, the received inpatient data into a plurality of Diagnosis Related Groups (DRG)s;
generating, by the computer processor, a plurality of clusters of physicians via a k-means method, wherein the plurality of clusters of physicians is based on a consumption of the one or more service matters for each of the plurality of DRGs, and wherein the plurality of clusters are generated iteratively by the computer processor to determine an optimal number of clusters based on an overall R-Square value, wherein a plurality of responsible physicians (RP)s with identical consumption across the one or more service matters within each of the plurality of DRGs are assigned to the same cluster;
on a condition that the received inpatient data contains cost information at a service item level, for each consumption of the one or more service matters with one of the plurality of DRGs, determining by the computer processor, a plurality of conflict indexes across the plurality of clusters of physicians based on a cost incurred for consumption of the one or more service matters;
receiving feedback from a remote user computer;
generating a visualization based on the feedback; and
transmitting the visualization to interactively display on the remote user computer display, in an interactive dashboard, the plurality of conflict indexes for each of the plurality of DRGs and the one or more service matters having the greatest potential for cost savings, wherein user interaction with the interactive dashboard displaying the plurality of conflict indexes causes display of the consumption across the one or more service matters for each of the plurality of discharges for each of the one or more clusters.

2. The method of claim 1, wherein the inpatient data is inpatient data for one hospital or inpatient data for a plurality of hospitals in a particular grouping.

3. The method of claim 1, wherein on a condition that the received inpatient data includes charge information and does not contain cost information at a service item level, the charge information is adjusted to costs using cost to charge ratios (RCC)s while excluding or correcting outlier RCCs and missing RCCs to form a costed discharge record as the received inpatient data.

4. The method of claim 1, wherein the RP is assigned to a cluster on a condition that the number of cases that the RP takes care for the same DRG is more than a threshold minimum cases number (nc), and on a condition that the number of RPs taking care of one DRG is more than the threshold minimum physicians number (np).

5. The method of claim 1 further comprising determining a potential cost savings opportunity wherein the potential cost savings opportunity is a variance between an actual cost and an optimal cost based on resource consumption.

6. The method of claim 5 further comprising interactively displaying the potential cost savings opportunities on the remote user computer display.

7. The method of claim 1 further comprising classifying the plurality of DRGs with an All Patient Refined Diagnosis Related Group (APR DRG).

8. The method of claim 1, wherein on a condition that the APR DRG is surgical, an RP is a first entry in another physician location of a Uniform Bill, and on a condition that the other physician location is empty, an attending physician is used as the RP, and on a condition that the attending physician is empty, no RP is assigned, or on a condition that the APR DRG is not surgical, the RP is an attending physician, and on a condition that the attending physician is empty, no RP is assigned.

9. The method of claim 1, wherein a conflict index value is calculated for each consumption dimension within one DRG as
$$Conflict Index = 1 - \frac{{\sum }_{{i = 1}_{c = 1}}^{{N}^{C}} {( yi - {\tilde{y}}_{c} )}^{2}}{{\sum }_{i = 1}^{N} {( yi - \overline{y} )}^{2}}$$
in which N is a number of RPs for the DRG, C is a number of clusters, within one of the DRGs for one consumption dimension, Yi is the average cost for RP, Y-bar is the mean of the average cost among all physicians for the DRG, Yc-hat is an average physician cost in cluster.

10. A system for optimization of medical resource consumption comprising:
a computer comprising a computer processor configured to receive inpatient data from a remote computer, wherein the inpatient data is directed to cost information for one or more service matters for a plurality of discharges, wherein the computer processor is further configured to classify, for each of the plurality of discharges, the received inpatient data into a plurality of Diagnosis Related Groups (DRG)s;
the computer processor configured to generate a plurality of clusters of physicians via a k-means method, wherein the plurality of clusters of physicians is based on a consumption of the one or more service matters for each of the plurality of DRGs, and generate the plurality of clusters iteratively to determine an optimal number of clusters based on an overall R-Square value, wherein a plurality of responsible physicians (RP)s with identical consumption across the one or more service matters within each of the plurality of DRGs are assigned to the same cluster and for each consumption of the one or more service matters with one of the plurality of DRGs;
on a condition that the received inpatient data contains cost information at a service matter level, determine a plurality of conflict indexes across the plurality of clusters based on a cost incurred for consumption of the one or more service matters;
the computer processor configured to receive feedback from a remote user computer;
the computer processor configured to generate a visualization based on the feedback; and
the computer processor configured to transmit the visualization to interactively display on the remote user computer display, in an interactive dashboard, the plurality of conflict indexes for each of the plurality of DRGs and the one or more service matters having the greatest potential for cost savings, wherein user interaction with the interactive dashboard displaying the plurality of conflict indexes causes display of the consumption across the one or more service matters for each of the plurality of discharges for each of the one or more clusters.

11. The system of claim 10, wherein the inpatient data is inpatient data for one hospital or inpatient data for a plurality of hospitals in a particular grouping.

12. The system of claim 10, wherein on a condition that the received inpatient data includes charge information and does not contain cost information at a service item level, the charge information is adjusted to costs using cost to charge ratios (RCC)s while excluding or correcting outlier RCCs and missing RCCs to form a costed discharge record as the received inpatient data.

13. The system of claim 10, wherein the RP is assigned to a cluster on a condition that the number of cases that the RP takes care for the same DRG is more than a threshold minimum cases number (nc), and on a condition that the number of RPs taking care of one DRG is more than the threshold minimum physicians number (np).

14. The system of claim 10, wherein the computer processor is further configured to determine a potential cost savings opportunity wherein the potential cost savings opportunity is a variance between an actual cost and an optimal cost based on resource consumption.

15. The system of claim 14, wherein the computer processor is further configured to interactively display the potential cost savings opportunities on the remote user computer display.

16. The system of claim 10, wherein the computer processor is further configured to classify the plurality of DRGs with an All Patient Refined Diagnosis Related Group (APR DRG).

17. The system of claim 10, wherein on a condition that the APR DRG is surgical, an RP is a first entry in another physician location of a Uniform Bill, and on a condition that the other physician location is empty, an attending physician is used as the RP, and on a condition that the attending physician is empty, no RP is assigned, or on a condition that the APR DRG is not surgical, the RP is an attending physician, and on a condition that the attending physician is empty, no RP is assigned.

18. The system of claim 10, wherein a conflict index value is calculated for each consumption dimension within one DRG as
$$Conflict Index = 1 - \frac{{\sum }_{{i = 1}_{c = 1}}^{{N}^{C}} {( yi - {\tilde{y}}_{c} )}^{2}}{{\sum }_{i = 1}^{N} {( yi - \overline{y} )}^{2}}$$
in which N is a number of RPs for the DRG, C is a number of clusters, within one of the DRGs for one consumption dimension, Yi is the average cost for RP, Y-bar is the mean of the average cost among all physicians for the DRG, Yc-hat is an average physician cost in cluster.
